(19) United States
(12) Patent Application Publication
(10) Pub. No.: US 2024/0384303 A1
(43) Pub. Date: Nov. 21, 2024

(54) GENOME EDITING METHOD BASED ON CRISPR/CAS9 SYSTEM AND USE THEREOF

(71) Applicant: CHUNGANG UNIVERSITY INDUSTRY ACADEMIC COOPERATION FOUNDATION, Seoul (KR)

(72) Inventors: Sang Jun Lee, Seoul (KR); Ho Joung Lee, Busan (KR); Hyun Ju Kim, Gumi-si (KR)

(21) Appl. No.: 17/995,223

(22) PCT Filed: Apr. 2, 2021
(86) PCT No.: PCT/KR2021/004155

§ 371 Date: Sep. 30, 2022

(30) Foreign Application Priority Data
Apr. 2, 2020 (KR) 1020200040352

(51) Int. Cl. C12N 15/90; C12N 9/22; C12N 15/11

(52) U.S. Cl. CPC C12N 15/902; C12N 9/22; C12N 15/11; C12N 2310/20

(57) ABSTRACT

The present invention pertains to a genome editing method based on a CRISPR/CAS9 system, and a use thereof. A CRISPR system using oligonucleotide-induced mutagenesis and mismatch guide RNA (sgRNA) according to the present invention achieves a significant genome editing effect on target DNA. Thus, it is expected that the CRISPR system of the present invention will be able to be used in a wide range of fields, such as compositions for gene editing using genetic scissors, genome level screening, therapeutic agents for treating various diseases including cancer, the development of compositions for disease diagnosis or imaging, and the development of transgenic plants and animals.

### INCORPORATION BY REFERENCE OF SEQUENCE LISTING

The content of the electronically submitted sequence listing, file name: Q285791_sequence listing as filed.TXT; size: 16,152 bytes; and date of creation: Apr. 11, 2023, filed herewith, is incorporated herein by reference in its entirety.

### TECHNICAL FIELD

The present invention relates to a method for genome editing based on a CRISPR/Cas9 system, and uses thereof.

### BACKGROUND ART

Since the birth of genome editing technology in 1970s, various gene editing tools have been developed. A Clustered Regularly Interspaced Short Palindromic Repeats (CRISPR) system, which is currently being actively studied as a genome editing tool, is a kind of adaptive immune system that microorganisms surviving after infection with bacteriophage, etc. store a part (about 20 bases) of an infected DNA sequence in the form of a spacer and then recognize the partial infected DNA when re-infected to cause the double-strand breakage in the infected DNA. Even among them, the CRISPR/Cas9 (CRISPR-associated 9) system requires only a single polypeptide to cause the double-stranded breakage at a target base site. Since the functions thereof are divided into guide RNA serving as a guide to the target base site, and Cas9 nuclease causing the double-strand breakage at the target site, respectively. Accordingly, as compared to previous generation genome editing tools, such as ZFN and TALEN, the CRISPR/Cas9 system has advantages of having a simple principle and low design cost and thus, has been the most widely used.

In order to cause the double-strand breakage at the target site, the CRISPR system requires an interaction between the Cas9 nuclease and a protospacer adjacent motif (PAM) adjacent to the target site in addition to complementary matching between the guide RNA and the target base. The PAM is a short sequence that exists right next to the target site, and becomes an important criterion for distinguishing foreign DNA from self-DNA in the CRISPR system. The CRISPR/Cas9 system has a PAM sequence of 5′-NGG, which acts as an obstacle that limits the range of sites that can be selected as targets in the genome. Accordingly, CRISPR/Cas systems with different PAM sequences have been studied to broaden the range that can be selected as targets.

Since the CRISPR/Cas9 system is derived from microorganisms, the CRISPR/Cas9 system has the activity to cause the double-strand breakage even if the sequence of the guide RNA does not fully match the target site. Since the CRISPR/Cas9 system is widely used even for the genome editing of eukaryotes as well as the microorganisms, the accuracy to cause the double-strand breakage only at the target site is critical. Accordingly, in order to reduce mismatch tolerance, research has been conducted on shortening the length of the guide RNA or changing the structure of the Cas9 nuclease.

Introduction of single-stranded oligonucleotide-induced mutagenesis, which introduces mutations by inserting DNA into cells, has a simple principle, but it was difficult to obtain a desired mutant due to a low yield, and in order to solve this problem, it has been used to edit the genome of microorganisms in association with the CRISPR/Cas9 system. Cells without mutagenesis in the target site are recognized as a target by CRISPR/Cas9 and die due to double-strand breakage in the cell genome, and the cells with mutagenesis in the target DNA sequence by the introduction of the oligonucleotide-induced mutagenesis are not recognized as a target but survive to obtain a mutated strain, which is referred to as negative selection, and it is possible to obtain genome-edited microbial mutants through negative selection.

The research team at the University of Wisconsin-Madison in the United States produced a genome-edited mutant strain from Lactobacillus reuteri using the CRISPR/Cas9 system and oligonucleotides containing a PAM site. The research team at Tianjin University in China induced gene deletions, point mutations, and codon mutations in a genome of Escherichia coli using the CRISPR/Cas9 system and the oligonucleotides. The research team at the Tianjin Institute of Industrial and Biotechnology in China succeeded in editing a genome of Corynebacterium glutamicum, which has been widely used as an industrial strain, using the CRISPR/Cas9 system and the oligonucleotides containing the PAM sequence.

As described above, interest has been focused on the study of editing the genome of microorganisms using the CRISPR/Cas9 system, but it is difficult to find prior studies that caused point mutations at a target site due to the mismatch tolerance of the CRISPR/Cas9 system, which recognizes and kills targets with point mutations of 1 to 2 bases identically to targets without mutations.

Accordingly, there is a need for research on a method that can overcome these obstacles and freely, easily and efficiently edit a genome of a target including microorganisms.

### PRIOR ARTS

### Patent Documents

US20170226522 A1
Korean Patent Publication No. 10-2016-0122197 (published on Oct. 21, 2016)

### Non-Patent Document

Li, Y., Lin, Z., Huang, C., Zhang, Y., Wang, Z., Tang, Y. J., Chen, T., Zhao, X., 2015. Metabolic engineering of Escherichia coli using CRISPR-Cas9meditated genome editing. Metab Eng. 31, 13-21.
Pyne, M. E., Moo-Young, M., Chung, D. A., Chou, C. P., 2015. Coupling the CRISPR/Cas9 System with Lambda RedRecombineering Enables Simplified Chromosomal Gene Replacement in Escherichia coli. Appl EnvironMicrobiol. 81, 5103-14.

### DISCLOSURE

### Technical Problem

Under such circumstances, the present inventors have made studies to develop a method capable of precisely editing a genome of a subject at a single base level based on a CRISPR/Cas9 system. Accordingly, the present inventors have designed the incorporation of a site-directed mutagenesis using oligonucleotides including a nucleotide sequence which is not perfectly complementary to target DNA, into CRISPR/Cas9 system including target-mismatched sgRNA introduced with a base sequence that is not complementary to the target DNA. As a result, the present inventors have found that where two or more mismatches were given (generated) between the target DNA and the guide RNA sequence, thereby overcome the mismatch tolerance of the CRISPR/Cas9 system, accurately edit (correct) a genome of E. coli to a single base level, and improve the point mutation incorporation efficiency, so that the present disclosure has been completed.

Therefore, an object of the present invention is to provide a genome editing method based on a CRISPR/Cas9 system.

Another object of the present invention is to provide a genome editing composition based on a CRISPR/Cas9 system.

Yet another object of the present invention is to provide a method for increasing genome editing efficiency based on a CRISPR/Cas9 system.

Still another object of the present invention is to provide a method for preparing a subject in which target DNA is edited based on a CRISPR/Cas9 system.

Still yet another object of the present invention is to provide a subject in which target DNA is edited, prepared by the method for preparing the subject in which the target DNA is edited based on the CRISPR/Cas9 system.

Other objects and advantages of the present invention will be more apparent by the following detailed description and claims.

### Technical Solution

The terms used herein are used for the purpose of description only, and should not be construed to be limited. The singular expression includes the plural expression unless the context clearly dictates otherwise. In the present application, it should be understood that term “comprise” or “have” indicates that a feature, a number, a step, an operation, a component, a part or the combination thereof described in the specification is present, but does not exclude a possibility of presence or addition of one or more other features, numbers, steps, operations, components, parts or combinations thereof, in advance.

Unless otherwise contrarily defined, all terms used herein including technological or scientific terms have the same meanings as those generally understood by a person with ordinary skill in the art. Terms which are defined in a generally used dictionary should be interpreted to have the same meaning as the meaning in the context of the related art, and are not interpreted as ideal or excessively formal meanings unless otherwise defined in the present application.

As used herein, the terms “nucleic acid sequence”, “nucleotide sequence” and “polynucleotide sequence” include: oligonucleotide or polynucleotide, and fragments or portions thereof, and DNA or RNA of genomic or synthetic origin, which may be a single-strand or double-strand, and refers to a sense or antisense strand.

Hereinafter, the present disclosure will be described in detail.

According to an aspect of the present invention, the present invention provides a genome editing method based on a CRISPR/Cas9 system, and the method comprises generating one or more mismatched nucleotides between target DNA and the guide RNA sequence by a donor nucleic acid molecule and the guide RNA that complementarily bind to the target DNA.

As used herein, the term “genome editing”, unless otherwise specified, refers to editing, restoring, modifying, losing and/or altering a gene function by deletion, insertion, substitution, etc. of a nucleic acid molecule by Cas9 cleavage at a target site of the target DNA.

Preferably, the present invention is characterized in that one or more mismatched nucleotides between the guide RNA and the target DNA rather enhance an editing effect of the CRISPR system.

The one or more mismatches between the guide RNA and the target DNA are achieved by introduction of a donor nucleic acid molecule and mismatch introduction of artificial guide RNA.

In the present invention, the term “donor nucleic acid molecule” or “donor nucleic acid sequence” as used in reference to genome editing based on the CRISPR/Cas9 system refers to a natural or modified polynucleotide, an RNA-DNA chimera, or a DNA fragment, or a PCR amplified ssDNA or dsDNA fragment or an analog thereof, including a target nucleotide sequence to be inserted into target DNA.

Such a donor nucleic acid molecule may include any form, such as single-stranded and double-stranded forms, as long as the donor nucleic acid molecule may cause genetic modifications on the target DNA to achieve the subject of the present invention.

The modifications on the target DNA may include a substitution of one or more nucleotides, an insertion of one or more nucleotides, a deletion of one or more nucleotides, a knockout, a knockin, a replacement of an endogenous nucleic acid sequence with a homologous, orthologous, or heterologous nucleic acid sequence, or a combination thereof.

In the present invention, preferably, in the modification on the target DNA, point mutations are introduced (induced) by substitution of one or more nucleotides in a wild-type DNA sequence, and the introduction of these point mutations is caused, for example, by oligonucleotides.

The introduction of the point mutations described above causes mismatches with the target DNA.

The length of the term “oligonucleotide” as used herein while referring to mutagenesis (induction) refers to a nucleic acid sequence of 10 to 90 nucleotides, preferably 15 to 85 nucleotides (mer), more preferably 20 to 50 nucleotides (which may be used as a probe or an amplimer). In an embodiment of the present invention, the mutagenic oligonucleotide was used with a length of 41 mer, but is not limited thereto as long as the object of the present invention may be achieved.

As used herein, the term “mismatch” refers to a state in which inappropriate base pairs occur, in which a non-complementary sequence exists in complementary bonds between DNAs or between DNA and RNA bases. The present inventors confirmed that CRISPR did not recognize the target DNA due to a mismatch existing by point mutation of one or more nucleotides in a wild-type DNA sequence and a mismatch occurring by generating intentionally mismatched bases between complementary guide RNAs to the target DNA, using the oligonucleotides and the guide RNA used for CRISPR/Cas.

Accordingly, the term “mismatched nucleotide”, “mismatched base”, or “mismatched nucleotide” as used herein refers to a non-complementary nucleotide and is used interchangeably with the non-complementary nucleotide.

In addition, as long as the object of the present invention can be achieved, the mismatched nucleotides may be located at various sites on the guide RNA.

That is, the site of the mismatched nucleotide on the guide RNA may exist at any site as long as the CRISPR does not recognize the target DNA, by providing artificial mismatched bases between the target DNA and the guide RNA complementary thereto.

According to the present invention, the site of the mismatched nucleotide may be located at an immediately adjacent site which is spaced apart by 1 nucleotide or a site far away by 10 or more nucleotides from the site on the guide RNA, corresponding to the site at which the point mutation on the donor DNA exists.

As long as the object of the present invention can be achieved, the number of mismatched nucleotides is not limited, but the number of mismatched nucleotides may be preferably 1 to 10, more preferably 1 to 5, much more preferably 1 to 3, even much more preferably 1 to 2, most preferably 1 (single mismatched nucleotide).

In addition, if there are at least two mismatched nucleotides in the guide RNA, the mismatched nucleotides may be located contiguously or discontinuously.

The term “immediately adjacent” as used herein while mentioning the mismatched nucleotide site refers to located at a site juxtapositioned in a 5′- or 3′-end direction, that is, spaced apart by 1 nucleotide from the site corresponding to a site where the point mutation exist, on a donor nucleic acid molecule (e.g., oligonucleotide) that causes the modification on the target DNA.

The term “guide RNA” refers to RNA specific to target DNA, may form a complex with a Cas protein, and refers to RNA that brings the Cas protein to the target DNA.

The guide RNA is dual RNA including CRISPR RNA (crRNA) and transactivating crRNA (tracrRNA) that hybridize with the target DNA, or single-chain guide RNA (sgRNA) including a part of the crRNA and tracrRNA and hybridizing with the target DNA, and in an embodiment of the present invention, the guide RNA is sgRNA.

Any guide RNA may be used in the present invention, as long as the guide RNA includes essential parts of the crRNA and the tracrRNA and a part complementary to the target.

The crRNA may hybridize with the target DNA.

The guide RNA may be transferred to cells or organisms in the form of RNA or DNA encoding the guide RNA. In addition, the guide RNA may also be a form of isolated RNA, RNA contained in a viral vector, or a form encoded in the vector. Preferably, the vector may be a viral vector, a plasmid vector, or an agrobacterium vector, but is not limited thereto.

In the present invention, the term “mismatch guide RNA” used while referring to the genome editing based on the CRISPR/Cas9 system is sgRNA including crRNA with one or more mismatched nucleotides between the target DNA and the guide RNA sequence, and is used interchangeably with target-mismatched sgRNA in this specification.

As used herein, the term “hybridization” means that complementary single-stranded nucleic acids form a double-stranded nucleic acid. The hybridization may occur even if complementarity between two nucleic acid strands is perfectly matched or some mismatched bases are present.

As used herein, the term “Cas protein” refers to an essential protein element in the CRISPR/Cas system, and forms active endonuclease or nickase when forming a complex with two RNAs called CRISPR RNA (crRNA) and trans-activating crRNA (tracrRNA).

Information on Cas gene and protein may be obtained from GenBank of the National Center for Biotechnology Information (NCBI), but is not limited thereto. A CRISPR-associated (cas) gene encoding the Cas protein is often associated with a CRISPR repeat-spacer array. The CRISPR-Cas system has two classes and six types, and among them, a type±ISPR/Cas system involving the Cas9 protein and the crRNA and tracrRNA is representatively well-known.

The target DNA includes a nucleotide of a complementary sequence to the crRNA or sgRNA and a protospacer-adjacent motif (PAM).

The oligonucleotide includes a protospacer-adjacent motif (PAM) of the target DNA.

The PAM is a 5′-NGG-3′ trinucleotide.

When a mutagenic oligonucleotide, which is the prior art, is inserted into cells, mutations are only introduced at a low yield in the process of replicating DNA. Even when using a conventional CRISPR/Cas9 system, it was difficult to introduce a single base point mutation due to the mismatch tolerance of CRISPR/Cas9.

Therefore, in order to solve the above problems, the present inventors introduced a point mutation into a target base of a target gene while overcoming the mismatch tolerance of CRISPR/Cas9 to design mismatch guide RNA which affected effectively achieving the editing effect, and applied the designed mismatch guide RNA to the CRISPR/Cas9 system together with the oligonucleotide-induced mutagenesis.

As a result, it was identified that due to the number of mismatched bases by point mutation and the number of mismatched bases increased by the mismatch guide RNA (target-mismatched sgRNA), genetic scissors did not recognize the target and thus the target DNA was not cleaved but survived, thereby achieving an effective gene editing effect.

Therefore, the method provided in the present invention may increase a genome editing effect compared to conventional guide RNA without mismatch.

According to an embodiment of the present invention, cells were inserted with an oligonucleotide causing a point mutation in 1 to 4 bases (504 to 507 positions) of a galK gene site which is a galactose sugar transporter gene to induce site-directed mutagenesis and then negatively selected by CRISPR/Cas9 and smeared on a McConkey plate selective medium. At this time, the editing efficiency was obtained as 89% when the mutation was induced in two bases, 94% when the mutation was induced in three bases, and 92% when the mutation was induced in four bases, but a strain in which a point mutation was introduced into a single base could not be obtained.

When the mismatched sequence is located immediately adjacent to the site on the guide RNA corresponding to the site of the base into which the mutation is introduced by the oligonucleotide, cells without mutagenesis die, and cells with mutagenesis survive due to the increased number of mismatched bases.

That is, the cells without mutagenesis die due to double-strand breakage in the target gene, and the cells with mutagenesis are not recognized as the target, but survive, which is called negative selection.

In addition, after mutagenesis at base 504 of a galK gene by oligonucleotide-induced mutagenesis, the negative selection was performed by the CRISPR/Cas9 system using single-base mismatch guide RNA to obtain strains in which a single base was mutated with the editing efficiency from a minimum of 36% to a maximum of 95%.

In addition, in the case of negative selection by the CRISPR/Cas9 system using double-base mismatch guide RNA, negative selection was impossible in all cases except for one case.

A part of a mutant progeny strain genome selected after the mutagenesis in the above-described manner was compared with a genome sequence of a parent strain after sequencing, and as a result, it was confirmed that a single base point mutation was introduced into a target base.

As a result, by the introduction of the oligonucleotide-induced mutagenesis and CRISPR/Cas9 negative selection using the mismatch guide RNA, it was possible to accurately introduce mutations only at a target base in an E. coli genome.

Therefore, the method of the present invention proves that a genome of an object may be efficiently and exquisitely edited as a single base unit by introducing a single base point mutation.

Further, according to another aspect of the present invention, the present invention provides a genome editing composition based on a CRISPR/Cas9 system comprising a donor nucleic acid molecule and guide RNA that complementarily bind to target DNA, to generate one or more mismatched nucleotides between the target DNA and the guide RNA sequence.

According to an exemplary embodiment of the present invention, the composition of the present invention recognizes the target gene in the CRISPR-Cas9 system, but includes a structure capable of expressing the target DNA and the guide RNA in which one or more mismatched sequences exist. The guide RNA and the donor DNA (e.g., oligonucleotide) are simultaneously transferred into the cells, and the donor DNA including the point mutation sequence is included in the genome instead of the target DNA through the donor DNA when the target DNA is cleaved by the Cas9 protein, thereby increasing the efficiency of substitution mutation of the target DNA.

Since the composition of the present invention uses the method of the present invention described above, the description of duplicated contents will be omitted in order to avoid excessive complexity of the present specification.

According to yet another aspect of the present invention, the present invention provides a method for increasing genome editing efficiency based on a CRISPR/Cas9 system, and the method comprises generating one or more mismatched nucleotides between the target DNA and the guide RNA sequence by a donor nucleic acid molecule and guide RNA, that complementarily bind to the target DNA.

According to an exemplary embodiment of the present invention, in the method for increasing the genome editing efficiency based on the CRISPR/Cas9 system of the present invention, a single point mutation is introduced in base pair alignment between a specific combination of target:guide RNA, that is, alignment of pyrimidine (Py:Py) base pairs or purine (Pu:Pu) base pairs, so that the editing efficiency is doubled.

Since the method of the present invention uses the method of the present invention described above, the description of duplicated contents will be omitted in order to avoid excessive complexity of the present specification.

Further, according to still another aspect of the present invention, the present invention provides a method for preparing a subject in which target DNA is edited based on a CRISPR/Cas9 system, comprising the steps of:
(a) constructing a donor nucleic acid molecule that complementarily binds to target DNA and causes modification on the target DNA;
(b) constructing guide RNA that complementarily binds to the target DNA and has one or more mismatched nucleotides generated to the target DNA; and
(c) contacting the donor nucleic acid molecule of step (a) and the guide RNA of step (b) into the subject to be edited, thereby two or more mismatches occur between the target DNA and the guide RNA sequence to edit the target DNA of the subject.

In addition, the CRISPR/Cas9 system of the present invention may use any selective marker known in the art as long as the object of the present invention may be achieved.

The object of the present invention is not limited as long as the method of the present invention may be applied, but may be preferably a plasmid, a virus, a prokaryotic cell, an isolated eukaryotic cell, or a eukaryotic organism other than a human.

The eukaryotic cell may be a cell of yeast, mold, plant, insect, amphibian, mammal, etc. For example, the eukaryotic cell may be a cell cultured in vitro, a transplanted cell, a primary cultured cell, an in vivo cell, or a mammal cell including a human, which is generally used in the art, but is not limited thereto.

In an exemplary embodiment of the present invention, since a strain in which the cas9 gene is integrated into the genome is used, the process of inserting the cas9 plasmid is unnecessary, and stable overexpression of the Cas9 protein may be induced. Since additional amplification and purification processes using dsDNA are not required and only an inserting process of an oligonucleotide containing a single point mutation and a guide RNA plasmid is required, the overall time for genome editing has been shortened.

Since the guide RNA plasmid used in an exemplary embodiment of the present invention is lost while cultured at 42° C., continuous genome editing is possible, and since the cas9 gene integrated into the genome is easily removed by a culture in a minimal medium containing P1 transduction and L-arabinose, it is possible to edit the genome without any trace so as not to be significantly affected by problems associated with genetically modified organisms.

The nucleic acid or Cas9 protein encoding the Cas9 protein may be any nucleic acid or Cas9 protein as long as the object of the present invention may be achieved, but is preferably derived from the genus Streptococcus.

According to still yet another aspect of the present invention, the present invention provides a subject in which the target DNA has been edited, prepared by the method as described above.

For example, the subject in which the target DNA has been edited is an E. coli MG1655 mutant strain in which a function of the gene is deleted or mutated by a single base point mutation occurring in specific bases of a yaaA gene, a ybdG gene, a vdcO gene, a ydiU gene, a preT gene, a ypdA gene, a fau gene, a vhbU gene, a mnmE gene, a thiH gene, a proX gene, a galK gene, a moeA gene, and a vjhF gene, prepared according to the method of the present invention.

The present invention relates to a method for editing and repairing a target genome in a single base unit, and has an effect of providing a method for producing a strain optimizing the production capacity of useful materials by correctly repairing a genome of a target object, for example, microbial strains, in which a target gene is mutated, or causing a codon change and the like.

### Advantageous Effects

According to the present invention, since the CRISPR system using the oligonucleotide-induced mutagenesis and the mismatch guide RNA (sgRNA) not only achieves a significant genome editing effect on target DNA, but also has a very small off-targeting effect, it is expected that the CRISPR system of the present invention will be able to be used in a wide range of fields, such as compositions for gene editing using genetic scissors, genome level screening, therapeutic agents for treating various diseases including cancer, the development of compositions for disease diagnosis or imaging, and the development of transgenic plants and animals.

### DESCRIPTION OF DRAWINGS

FIG. 1 illustrates a schematic diagram of the contents and basic principles of the present invention.

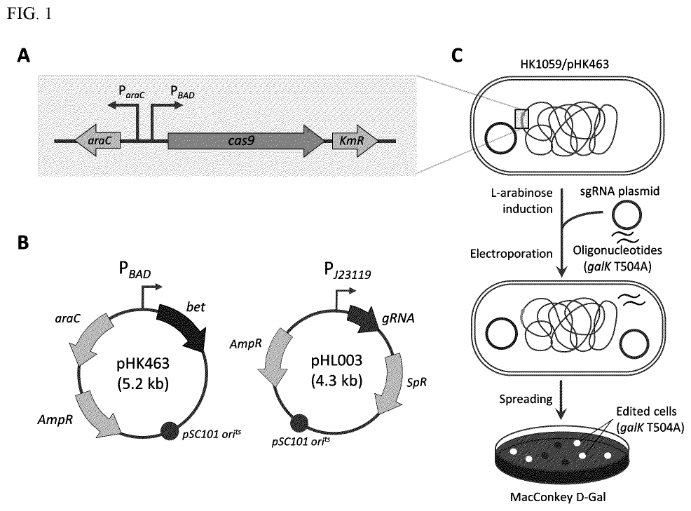

FIG. 2 shows a graph of editing efficiency and a colony forming unit by length of mutagenesis of a CRISPR/Cas9 system through negative selection by the CRISPR/Cas9 system after oligonucleotide-induced mutagenesis that causes point mutations of various lengths, and illustrates a schematic diagram for mismatch tolerance in which a target with a point mutation is recognized the same as a target without a mutation.

FIG. 3 illustrates a schematic diagram of single base editing overcoming mismatch tolerance characteristics using mismatch guide RNA of CRISPR/Cas9, and shows a graph of editing efficiency and operation according to a mismatched sequence site of guide RNA when a single base point mutation is introduced using base mismatch guide RNA far from a PAM sequence during negative selection by a CRISPR/Cas9 system after oligonucleotide-induced mutagenesis.

FIG. 4 shows a graph of editing efficiency and operation according to a mismatched sequence site of guide RNA when a single base point mutation is introduced using mismatch guide RNA close to a PAM sequence during negative selection by the CRISPR/Cas9 system after oligonucleotide-induced mutagenesis.

FIG. 5 illustrates a schematic diagram of the contents of removing a lambda-red beta expression plasmid, a guide RNA plasmid, and a cas9 gene from a genome-edited strain.

FIG. 6 illustrates a schematic diagram and a result graph of point mutation introduction using single base mismatch guide RNA in 16 different targets in an E. coli MG1655 genome.

FIG. 7 shows introduction of xylR single point mutation using mismatch guide RNA/Cas9 of the present invention.

FIG. 8 illustrates a result of confirming a consumption rate level of xylose sugar in E. coli introduced with xylR single point mutation using the mismatch guide RNA/Cas9 of the present invention.

### MODES OF THE INVENTION

Hereinafter, Examples are to describe the present invention in more detail, and it will be apparent to those skilled in the art that the scope of the present invention is not limited by these Examples in accordance with the gist of the present invention.

### Example 1. Preparation of Strain in Which Cas9 Gene is Inserted Into Genome

The present inventors prepared a mutant E. coli strain (E. coli MG1655 araBAD::P_{BAD}-cas9-KmR), in which a cas9 gene was inserted into a genome, through lambda-red recombineering. An E. coli MG1655 strain was smeared on an LB solid medium, and then a grown single colony was inoculated into 200 ml of an LB liquid medium and cultured at 37° C. until OD₆₀₀ₙₘ became 0.4, centrifuged at 3500 rpm for 20 minutes and then washed twice with 40 ml of 10% glycerol to prepare electrocompetent cells.

The cas9 gene to be inserted was amplified by PCR from pCas (Addgene plasmid #62225), and amplified by PCR with a cas9-KmR cassette to have a homologous sequence for recombination together with a kanamycin gene. The amplified PCR product was purified and then inserted into E. coli MG1655 in which a lambda-red recombinant enzyme of a pKD46 plasmid was overexpressed by L-arabinose and located at the back of a promoter in which the expression of the gene was induced by L-arabinose to prepare a HK1059 strain.

A lambda-red beta expression plasmid pHK463 to aid recombination by oligonucleotides was prepared through isothermal assembly by amplifying pKD46 backbone and bet gene parts by PCR, respectively, and made to express a beta protein only upon induction of L-arabinose.

### Example 2. Genome Editing Using CRISPR-Cas9 Negative Selection and Oligonucleotide-Induced Mutagenesis

The HK1059 strain of Example 1 was smeared on an LB solid medium, and then a grown single colony was inoculated into 200 ml of an LB liquid medium and cultured at 37° C. until the OD₆₀₀ₙₘ became 0.4, centrifuged at 3500 rpm for 20 minutes and then washed twice with 40 ml of 10% glycerol to prepare electrocompetent cells.

To assist recombination by oligonucleotides, the HK1059 strain was inserted with the lambda-red beta expression plasmid pHK463 and smeared, and then a formed single colony was inoculated into 200 ml of an LB liquid medium and cultured at 30° C. until OD₆₀₀ₙₘ became 0.4, and additionally cultured for 3 hours after adding L-arabinose at a concentration of 1 mM to overexpress a lambda-red beta protein and a Cas9 protein. Thereafter, the HK1059 strain was centrifuged at 3500 rpm for 20 minutes, washed twice with 40 ml of 10% glycerol to prepare electrocompetent cells.

Mutagenesis oligonucleotides were prepared so that 1 to 4 bases were substituted from bases 503 to 507 of a galK gene (NCBI accession no. 945358), respectively. The HK1059 was inserted with a guide RNA-expressing plasmid and oligonucleotides by electroporation, and then smeared on a McConkey plate selective medium added with 5 g/L of galactose, and then cultured at 30° C.

When a mutation was introduced into a galK gene by the oligonucleotides, the galK gene was not normally expressed, making galactose metabolism impossible, and as a result of the inability to galactose metabolism, white colonies were formed in the McConkey plate selective medium. Since the mutation did not occur, the galactose metabolism was performed, and when the pH of the McConkey plate medium was lowered to 6.8 or less by metabolites, red colonies were formed. The editing efficiency of the CRISPR/Cas9 system was calculated by a color ratio [white colony/(white colony+red colony)] of colonies formed in the solid medium.

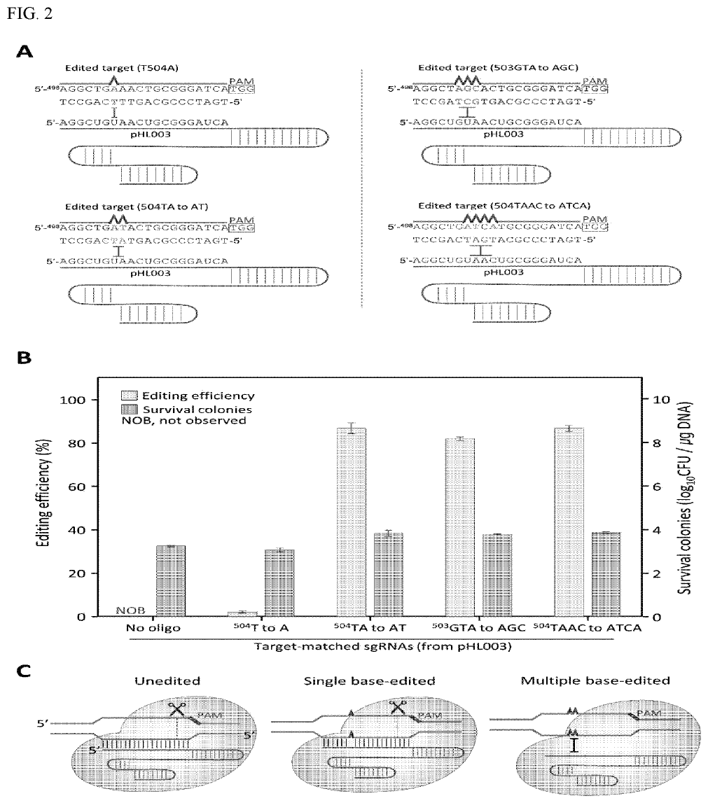

As a result, as illustrated in FIG. 2, a point mutation of two bases was introduced at the editing efficiency of 86%, a point mutation of three bases was introduced at the editing efficiency of 81%, and a point mutation of four bases was introduced at the editing efficiency of 86%. However, the editing efficiency of the introduction of the single point mutation was shown to be 2% due to the mismatch tolerance of the CRISPR/Cas9 system.

Accordingly, in order to overcome the mismatch tolerance of the CRISPR/Cas9 and precisely edit the genome at a single base level, the present inventors established a CRISPR/Cas9 system using mismatch guide RNA designed so that there were oligonucleotide-directed mutagenesis; and a mismatched sequence at a specific site so that a target single point mutation was introduced into the genome.

More specifically, the present inventors designed mismatch guide RNA so that the guide RNA had a base sequence that was not complementary to a target gene sequence to which the guide RNA was complementarily bound, that is, a mismatched sequence.

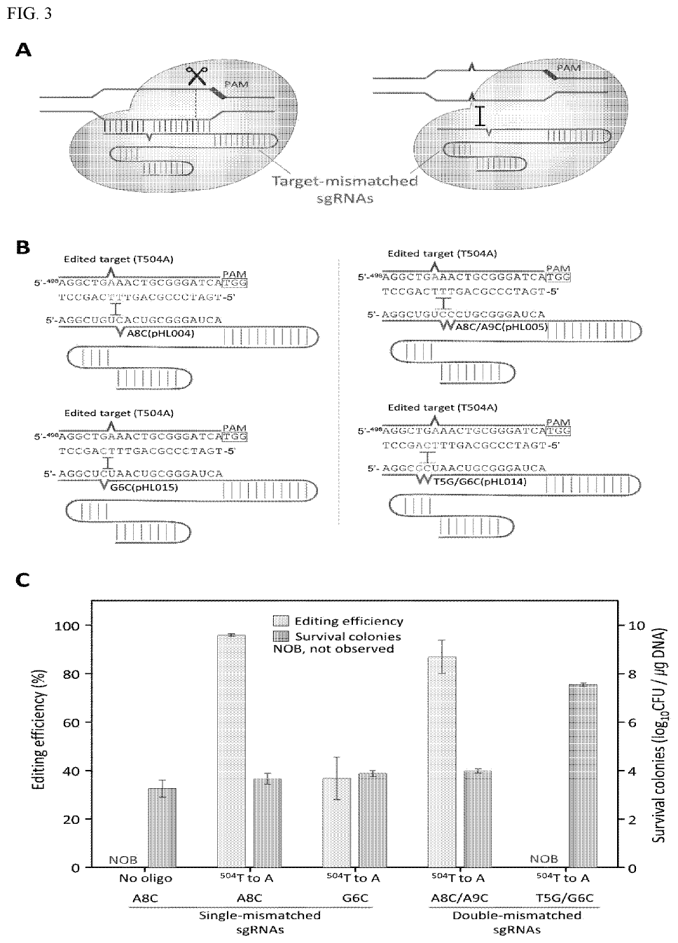

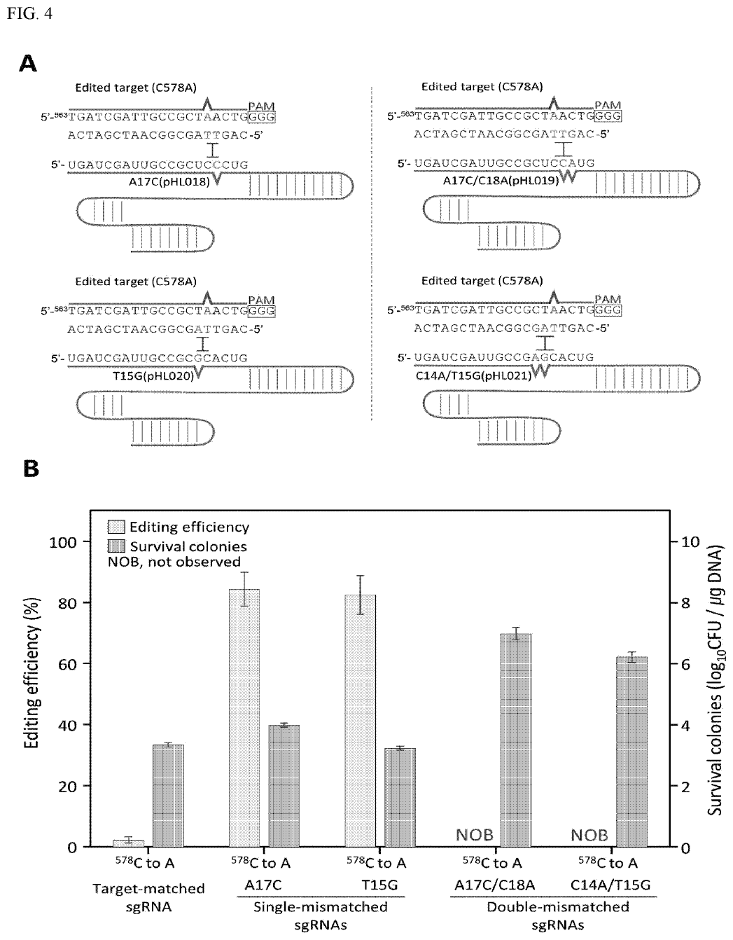

Accordingly, a total of two mismatches, one mismatched base with the target DNA by introduction of the point mutation and one mismatched base imparted on the guide RNA, were generated (FIGS. 3A, 3B and 4A).

Thereafter, when a mutation was introduced into a target DNA sequence of genetic scissors by oligonucleotides, the sequence in which the mutation was introduced was not recognized as a target sequence by the genetic scissors, so that cells may survive. However, since double-stranded DNA of a target in which the mutation was not introduced was cleaved by the mismatch tolerance of genetic scissors and then the cells did not survive (negative selection), a target genome may be efficiently edited and selected at a single base level.

### Example 3. Confirmation of Introduction Efficiency of Single Point Mutation According to Location and Number of Base Mismatches in Mismatch Guide RNA (Target-Mismatched sgRNA)

### 3-1. Location and Number of Mismatched Sequences on Guide RNA for Target Point Mutation Present at a Site Spaced Apart from PAM Sequence

In order to determine whether the location and number of mismatched sequences on mismatch guide RNA affected the editing efficiency, the present inventors introduced a single point mutation using an oligonucleotide in which a target point mutation was located at a site spaced (far) from the PAM sequence; and guide RNA having single or double mismatched sequences on both 5′ and 3′ based on the point mutation site, respectively.

This will be briefly described as follows.

An oligonucleotide causing a point mutation (T→A) at base 504 of a galK gene, which was a site spaced apart in a 5′-direction from the PAM sequence and guide RNA having single or double mismatched sequences on both 5′ and 3′ based on the point mutation site, respectively, were inserted into the strain overexpressed with the lambda-red beta protein and the Cas9 protein of Example 2, and then the editing efficiency and colony forming units were calculated in the same manner as in Example 2.

As a result, as illustrated in FIG. 3C, when a guide RNA plasmid having a single mismatched sequence at base 505 of the galK gene was used, the editing efficiency was about 95%. On the other hand, when there was a single mismatched sequence at base 503, the editing efficiency was reduced to 36%.

In addition, when a guide RNA plasmid having a double mismatched sequence at bases 505 and 506 of the galK gene was used, the editing efficiency was about 86%. On the other hand, when there was a double mismatched sequence at bases 502 and 503, white colonies were not formed and thus, the editing efficiency could not be calculated, and the colony forming unit value increased greater than before. The reason was that when there was the double mismatched sequence at bases 502 and 503, the guide RNA did not recognize a galK target gene sequence which was complementary bound thereto regardless of the introduction of the point mutation and thus, the genetic scissors did not operate, so that the cells were not negatively selected, but survived.

### 3-2. Location and Number of Mismatched Sequences on Guide RNA for Target Point Mutation Adjacent to PAM Sequence

In order to determine whether the location and number of mismatched sequences on mismatch guide RNA (target-mismatched sgRNA) affected the editing efficiency, the present inventors introduced a single point mutation using an oligonucleotide in which a target point mutation was located at a site adjacent (close) to the PAM sequence; and guide RNA having single or double mismatched sequences on both 5′ and 3′ based on the point mutation site, respectively.

This will be briefly described as follows.

In the same manner as in Example 3, an oligonucleotide causing a point mutation (C→A) at base 578 of the galK gene, which was a site adjacent to the PAM sequence and guide RNA having single or double mismatched sequences on both 5′ and 3′ based on the point mutation site, respectively, were inserted into the strain overexpressed with the lambda-red beta protein and the Cas9 protein of Example 2, and then the editing efficiency and colony forming units were calculated.

As a result, as illustrated in FIG. 4B, the editing efficiency was 84% when a single mismatched sequence was present at base 579 of the galK gene, and 82% when a mismatched sequence was present at base 577 of the galK gene.

On the other hand, when there were double mismatched sequences at bases 576 and 577 and bases 579 and 580, like the case of Example 3 above, white colonies were not formed and thus, the editing efficiency could not be calculated, and the colony forming unit value increased greater than before. The reason was that when there were the double mismatched sequences at bases 576 and 577 and bases 579 and 580, the guide RNA did not recognize the galK target gene sequence which was complementary bound thereto regardless of the introduction of the point mutation and thus the genetic scissors did not operate, so that the cells were not negatively selected, but survived.

That is, for the double mismatched sgRNA, no white colonies were observed and the survival rate was remarkably high, which indicated that a Cas9/double mismatched sgRNA complex could not recognize a non-edited target.

The results of Examples 3-1 and 3-2 showed that in the location of the mismatched sequence on the guide RNA, it was effective when there was at least one mismatched sequence at a site spaced apart by 1 nucleotide from the site on the guide RNA corresponding thereto, based on the site at which the target point mutation was introduced, regardless of the PAM sequence site such as whether the point mutation was spaced apart from or adjacent to the PAM sequence, in the CRISPR/Cas9 system using the oligonucleotide-directed mutagenesis and the mismatch guide RNA (target-mismatched sgRNA) of the present invention.

### Example 4. Removal of cas9 Gene Guide RNA Plasmid from Genome-Edited Strain

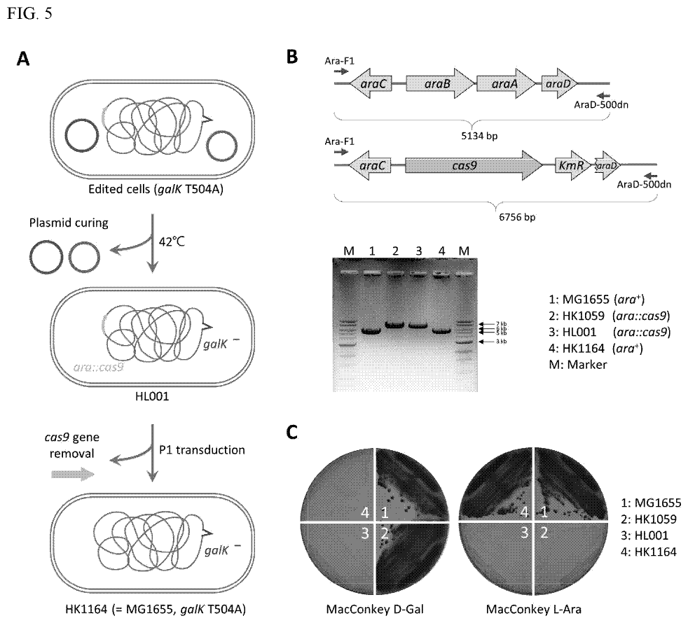

The present inventors removed the guide RNA plasmid by culturing the genome-edited strain at 42° C. to enable continuous genome editing at different sites. The cas9 gene was substituted with an araBAD gene through P1 bacteriophage transduction to obtain a strain in which only a single base point mutation occurred in the galK gene as compared with an original strain (FIG. 5).

### Example 5. Single Base Editing of 16 Different Targets Using Single Mismatch Guide RNA

The present inventors additionally verified whether the CRISPR/Cas9 system using the oligonucleotide-induced mutagenesis; and the mismatch guide RNA designed to have a mismatch at a specific site of the present invention was operated even at different target sites.

Therefore, 16 different CRISPR/Cas9 target sites were selected in a genome of an E. coli MG1655 strain, three oligonucleotides each causing point mutations different from the existing point mutations at nucleotide 11 of a target nucleotide sequence were prepared, and four guide RNAs per target site were prepared to include different three mismatched sequences in nucleotide 12 of the guide RNA.

The mutagenic oligonucleotides were prepared to substitute base 740 of a yaaA gene, base 685 of a ybdG gene, base 755 of a ydcO gene, base 285 of a ydiU gene, base 1125 of a preT gene, base 749 of a ypdA gene, base 371 of a fau gene, base 239 of a vhbU gene, base 39 of a mnmE gene, base 305 of a thiH gene, base 741 of a proX gene, bases 504, 578, and 935 of a galK gene, base 350 of a moeA gene, base 492 of a yjhF gene with three different bases (A→G/T/C, T→G/A/C, G→A/T/C, or C→G/A/T).

HK1059 was inserted by electroporation with a plasmid expressing guide RNA matching a target sequence per target site and three different guide RNAs each having a mismatched sequence on the right based on a point mutation site and point mutagenic oligonucleotides (16 targets×3 point mutagenic oligonucleotides×4 guide RNAs=192 electroporations) and then smeared on an LB medium and cultured at 37° C., and three colonies were randomly selected from the formed colonies, and the introduction of mutations was confirmed through Sanger sequencing.

As a result, among 48 possible point mutations (16 targets×3 point mutagenic oligonucleotides), 54% (26/48) of the point mutations were successfully induced in 13 targets, and mutation introduction was successful in 42 of the 192 electroporations.

On the other hand, the point mutation refers to a phenomenon in which a sequence is changed due to a mutation of one base pair, and includes Transition and Transversion. Transition is a phenomenon in which Py bases are converted to Py bases and Pu bases are converted to Pu bases, and Transversion is a phenomenon in which Py bases are converted to Pu bases or Pu bases are converted to Py bases (Py=pyrimidine=C or T, Pu=purine=G or A).

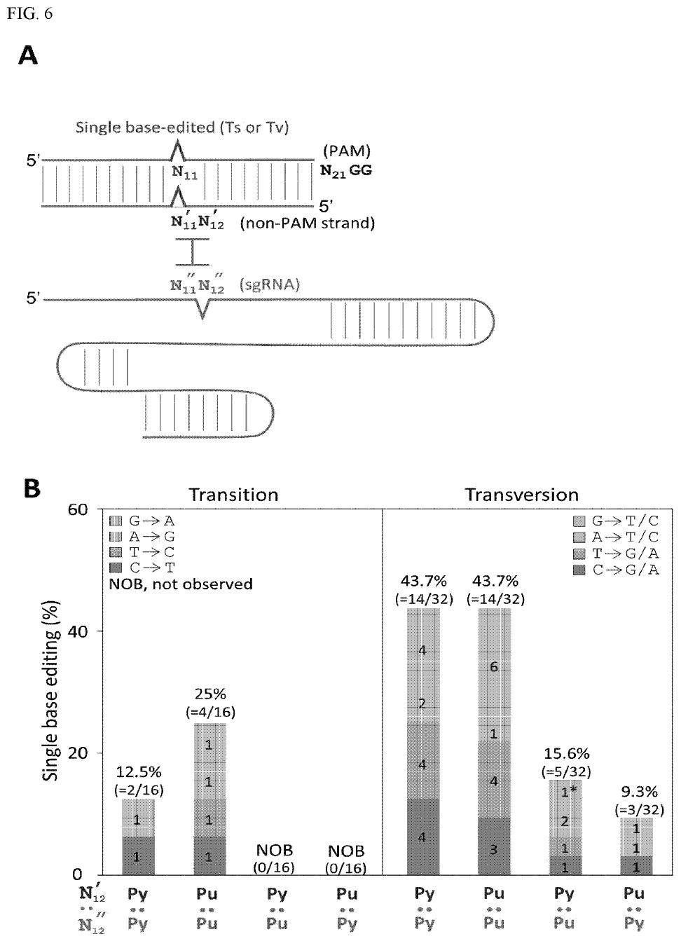

When the results of 42 electroporations (/192 electroporations) that were successful in mutagenesis were divided according to these mutation types (64 Transition+128 Transversion), Transition was 9% (6/64) and Transversion was more dominant at 28% (36/128). Only one of 42 electroporations was successful using target-matched guide RNA, but in case of Transition, N′₁₁:N′₁₁ corresponds to a combination of Py:Pu or Pu:Py, whereas in the case of Transversion, N′₁₁:N′₁₁ has a base pair alignment of Py:Py or Pu:Pu (FIG. 6B).

When the 42 electroporation results were divided according to the base pair alignment between the target and the guide RNA by determining that the base pair alignment affected the mismatch between the guide RNA and the target gene sequence to which the guide RNA was complementarily bound (FIG. 6B), in the case of Transversion, 43.7% of the point mutations were introduced under the base pair alignment of Py:Py or Pu:Pu, whereas the success rate was decreased in the base pair alignment of Py:Pu (15.6%) or Pu:Py (9.3%) including the target-matched guide RNA.

In addition, in the case of Transition, no point mutation was introduced when the base pair alignment of Py:Pu or Pu:Py was used, and the success rate of Py:Py was 12.5% and the success rate of Pu:Pu was 25%.

That is, there are effective purine or pyrimidine base pair moieties. The purine or pyrimidine base pair moiety is typically adenyl, cytosine, guanine, uracil or thymine.

This means that the mismatch guide RNA (sgRNA) of the present invention may not only overcome the mismatch tolerance of CRISPR/Cas9 and operate to increase the point mutagenesis efficiency, but also verify a specific base pair alignment condition between the mismatch guide RNA and the target gene sequence which was complementarily bound thereto, that is, an optimal condition capable of more effectively causing point mutations when a point mutation was introduced under the base pair alignment of Py:Py or Pu:Pu.

TABLE 1

| | | SEQ ID |
|---|---|---|
| Primer name | Primer base sequence (5′→3′) | NO: |
| Cas-AraC-F1 | GCTTTTTATCGCAACTCTCTACTGTTTCTCCATACCCGTTTTTTTGGATGGAGTG | 1 |
| | AAACGATGGATAAGAAATACTCAATAGGCT | |
| KmR-AraD-R4 | GTGGTGCCGGTTGCTGGAATCGACTGACCCGCCTGCGCCCAGATGGTGGCGTGGC | 2 |
| | GCGAGTGTAGGCTGGAGCTGCTTCGAAGTT | |
| Cas-KmR-OR2 | GACGGATCCCCGGAATTCAGTCACCTCCTAGCTGACTCAAA | 3 |
| Cas-KmR-OF3 | GCTAGGAGGTGACTGAATTCCGGGGATCCGTCGACCTGCAG | 4 |
| Ara-F1 | AGCAGCTCCGAATAGCGCCCTTCCCCTTGC | 5 |
| AraD_500dn | CCAGCCAGAAGGAGACTTCTGTCCCTTG | 6 |
| bet_for | AGGAGGTTATAAAAAATGAGTACTGCACTCGCAACGCTGGC | 7 |
| bet_rev | TTATCGTGAGGATGCGTCATGCTGCCACCTTCTGCTCTGCGG | 8 |
| rep101_for | AAGGTGGCAGCATGACGCATCCTCACGATAATATCCGGGTAG | 9 |
| ara_promoter_ | GAGTGCAGTACTCATTTTTTATAACCTCCTTAGAGCTCGAATTC | 10 |
| rcv | | |
| galK504A | GAAAACCAGTTTGTAGGCTGAAACTGCGGGATCATGGATCA | 11 |
| galK504AT | GAAAACCAGTTTGTAGGCTGATACTGCGGGATCATGGATCA | 12 |
| galK503AGC | GAAAACCAGTTTGTAGGCTAGCACTGCGGGATCATGGATCA | 13 |
| galK504ATCA | AAAACCAGTTTGTAGGCTGATCATGCGGGATCATGGATCAG | 14 |
| galK578A | CTTGCTGATCGATTGCCGCTAACTGGGGACCAAAGCAGTTT | 15 |
| Sm_ATG_Out | GATACTGGGCCGGCAGGCGCTCCATTGCCC | 16 |
| Sm_TAA_Out | GCAATGGAGCGCCTGCCGGCCCAGTATCAG | 17 |
| ClaI-gRNA-F | ATCGATAGCGAGTCAGTGAGCGAGGAAGCGGAAG | 18 |
| NcoI-gRNA-R | CCATGGGAAGATCCTTTGATCTTTTCTACGGGGTC | 19 |
| Cas-galK-WF | AGGCTGTAACTGCGGGATCAGTTTTAGAGCTAGAAATAGCAAG | 20 |
| Cas-galK-WR | TGATCCCGCAGTTACAGCCTACTAGTATTATACCTAGGACTG | 21 |
| galK505C_F | AGGCTGTCACTGCGGGATCAGTTTTAGAGCTAGAAATAGCAAG | 22 |
| galK505C_R | TGATCCCGCAGTGACAGCCTACTAGTATTATACCTAGGACTG | 23 |
| galK505CC_F | AGGCTGTCCCTGCGGGATCAGTTTTAGAGCTAGAAATAGCAAG | 24 |
| galK505CC_R | TGATCCCGCAGGGACAGCCTACTAGTATTATACCTAGGACTG | 25 |
| galK502GC_F | AGGCGCTAACTGCGGGATCAGTTTTAGAGCTAGAAATAGCAAG | 26 |
| galK502GC_R | TGATCCCGCAGTTAGCGCCTACTAGTATTATACCTAGGACTG | 27 |
| galK503C_F | AGGCTCTAACTGCGGGATCAGTTTTAGAGCTAGAAATAGCAAG | 28 |
| galK503C_R | TGATCCCGCAGTTAGAGCCTACTAGTATTATACCTAGGACTG | 29 |
| galK563_F | TGATCGATTGCCGCTCACTGGTTTTAGAGCTAGAAATAGCAAG | 30 |
| galK563_R | CAGTGAGCGGCAATCGATCAACTAGTATTATACCTAGGACTG | 31 |
| galK579C_F | TGATCGATTGCCGCTCCCTGGTTTTAGAGCTAGAAATAGCAAG | 32 |
| gal579C_R | CAGGGAGCGGCAATCGATCAACTAGTATTATACCTAGGACTG | 33 |
| galK579CA_F | TGATCGATTGCCGCTCCATGGTTTTAGAGCTAGAAATAGCAAG | 34 |
| galK579CA_R | CATGGAGCGGCAATCGATCAACTAGTATTATACCTAGGACTG | 35 |
| galK577G_F | TGATCGATTGCCGCGCACTGGTTTTAGAGCTAGAAATAGCAAG | 36 |
| galK577G_R | CAGTGCGCGGCAATCGATCAACTAGTATTATACCTAGGACTG | 37 |
| galK576AG_F | TGATCGATTGCCGAGCACTGGTTTTAGAGCTAGAAATAGCAAG | 38 |
| galK576AG_R | CAGTGCTCGGCAATCGATCAACTAGTATTATACCTAGGACTG | 39 |

TABLE 2

| Target gene | | |
|---|---|---|
| (target base | | SEQ ID |
| sequence) | Primer base sequence ((5′→3′) | NO: |
| yaaA | TTGATGAAGATTCCTCCAGCG*ATGGCGAACTGGTGTTTAAA | 40 |
| (TTCCTCCAGCA* | TTGATGAAGATTCCTCCAGCT*ATGGCGAACTGGTGTTTAAA | 41 |
| ATGGCGAAC) | TTGATGAAGATTCCTCCAGCC*ATGGCGAACTGGTGTTTAAA | 42 |
| galK | TCGAAATCACCGTGCCGCAAG*TTGACACTCTGGTAGAAATC | 43 |
| (CGTGCCGCAAA* | TCGAAATCACCGTGCCGCAAT*TTGACACTCTGGTAGAAATC | 44 |
| TTGACACTC) | TCGAAATCACCGTGCCGCAAC*TTGACACTCTGGTAGAAATC | 45 |
| ybdG | ATTGGGTTAACCACCGTCAAG*GTGCGTAACTGGGACAATAC | 46 |
| (CCACCGTCAAA* | ATTGGGTTAACCACCGTCAAT*GTGCGTAACTGGGACAATAC | 47 |
| GTGCGTAAC) | ATTGGGTTAACCACCGTCAAC*GTGCGTAACTGGGACAATAC | 48 |
| moeA | CGGTGGTGATGCAGGAGCAGG*CTGAACAAATGGACAATGGC | 49 |
| GCAGGAGCAGA* | CGGTGGTGATGCAGGAGCAGT*CTGAACAAATGGACAATGGC | 50 |
| CTGAACAAA) | CGGTGGTGATGCAGGAGCAGC*CTGAACAAATGGACAATGGC | 51 |
| galK | GAAAACCAGTTTGTAGGCTGG*AACTGCGGGATCATGGATCA | 52 |
| (AGGCTGT* | GAAAACCAGTTTGTAGGCTGA*AACTGCGGGATCATGGATCA | 53 |
| AACTGCGGGATCA) | GAAAACCAGTTTGTAGGCTGC*AACTGCGGGATCATGGATCA | 54 |
| ydcO | CATTAATTGTATTTACTGGAG*TGCTGGCACTGGTTTTTTCC | 55 |
| (ATTTACTGGAT* | CATTAATTGTATTTACTGGAA*TGCTGGCACTGGTTTTTTCC | 56 |
| TGCTGGCAC) | CATTAATTGTATTTACTGGAC*TGCTGGCACTGGTTTTTTCC | 57 |
| ydiU | TTGTAGTGCCATCAGCAAGCG*GTTGTTCGCCGAGTAAAATG | 58 |
| (GGCGAACAACT* | TTGTAGTGCCATCAGCAAGCT*GTTGTTCGCCGAGTAAAATG | 59 |
| GCTTGCTGA) | TTGTAGTGCCATCAGCAAGCC*GTTGTTCGCCGAGTAAAATG | 60 |
| preT | CCAGACCCGCGACCCGCAAGG*CTTTGCTGCTGGCGATATTG | 61 |
| (GACCCGCAAGT* | CCAGACCCGCGACCCGCAAGA*CTTTGCTGCTGGCGATATTG | 62 |
| CTTTGCTGC) | CCAGACCCGCGACCCGCAAGC*CTTTGCTGCTGGCGATATTG | 63 |
| ypdA | CGGTGGCGATTACTAATACCA*ATCATGTGCTGGCCTATGTT | 64 |
| (TACTAATACCG* | CGGTGGCGATTACTAATACCT*ATCATGTGCTGGCCTATGTT | 65 |
| ATCATGTGC) | CGGTGGCGATTACTAATACCC*ATCATGTGCTGGCCTATGTT | 66 |
| proX | CGATACCAAACTGCCGAATGA*TGCGAATTATGGCTTCCCGG | 67 |
| (CTGCCGAATGG* | CGATACCAAACTGCCGAATGT*TGCGAATTATGGCTTCCCGG | 68 |
| TGCGAATTA) | CGATACCAAACTGCCGAATGC*TGCGAATTATGGCTTCCCGG | 69 |
| fau | TGGTCGCCTTTGATGAGTACA*GTCAGCGCCTGGGAATGGGC | 70 |
| (TGATGAGTACG* | TGGTCGCCTTTGATGAGTACT*GTCAGCGCCTGGGAATGGGC | 71 |
| GTCAGCGCC) | TGGTCGCCTTTGATGAGTACC*GTCAGCGCCTGGGAATGGGC | 72 |
| yhbU | GTTACGCCCGTTGGCAGCGCA*CCGTGGATATGGCGGCGCAG | 73 |
| (TTGGCAGCGCG* | GTTACGCCCGTTGGCAGCGCT*CCGTGGATATGGCGGCGCAG | 74 |
| CCGTGGATA) | GTTACGCCCGTTGGCAGCGCC*CCGTGGATATGGCGGCGCAG | 75 |
| galK | CTTGCTGATCGATTGCCGCTG*ACTGGGGACCAAAGCAGTTT | 76 |
| (TGATCGATTGCCGCTC* | CTTGCTGATCGATTGCCGCTA*ACTGGGGACCAAAGCAGTTT | 77 |
| ACTG) | CTTGCTGATCGATTGCCGCTT*ACTGGGGACCAAAGCAGTTT | 78 |
| mnmE | TATCGTAGCCCAGGCCACGCG*TCCGGGACGTGGCGGCGTTG | 79 |
| (CAGGCCACGCC* | TATCGTAGCCCAGGCCACGCA*TCCGGGACGTGGCGGCGTTG | 80 |
| TCCGGGACG) | TATCGTAGCCCAGGCCACGCT*TCCGGGACGTGGCGGCGTTG | 81 |
| thiH | CCATGAGTAATCGCATCAAGG*GCAAAACGCTGGATGAAGCG | 82 |
| (TCGCATCAAGC* | CCATGAGTAATCGCATCAAGA*GCAAAACGCTGGATGAAGCG | 83 |
| GCAAAACGC) | CCATGAGTAATCGCATCAAGT*GCAAAACGCTGGATGAAGCG | 84 |
| yjhf | CCCCGGCCCGACTGTTATCGG*CAGGGAATATGGCGCTGATG | 85 |
| (ACTGTTATCGC* | CCCCGGCCCGACTGTTATCGA*CAGGGAATATGGCGCTGATG | 86 |
| CAGGGAATA) | CCCCGGCCCGACTGTTATCGT*CAGGGAATATGGCGCTGATG | 87 |

PAM sequences are indicated by underlined regions.
Single base mutations are indicated in bold *.

### Example 6. Verification of CRISPR/Cas9 System Using Oligonucleotide-Induced Mutagenesis and Mismatch Guide RNA of the Present Invention

The present inventors verified whether the CRISPR/Cas9 system using the oligonucleotide-induced mutagenesis designed so that the target single point mutation of the present invention was introduced; the mismatch guide RNA designed so that mismatches existed in a specific site was actually operated to overcome the mismatch tolerance of CRISPR/Cas9 and precisely edit the genome at a single base level.

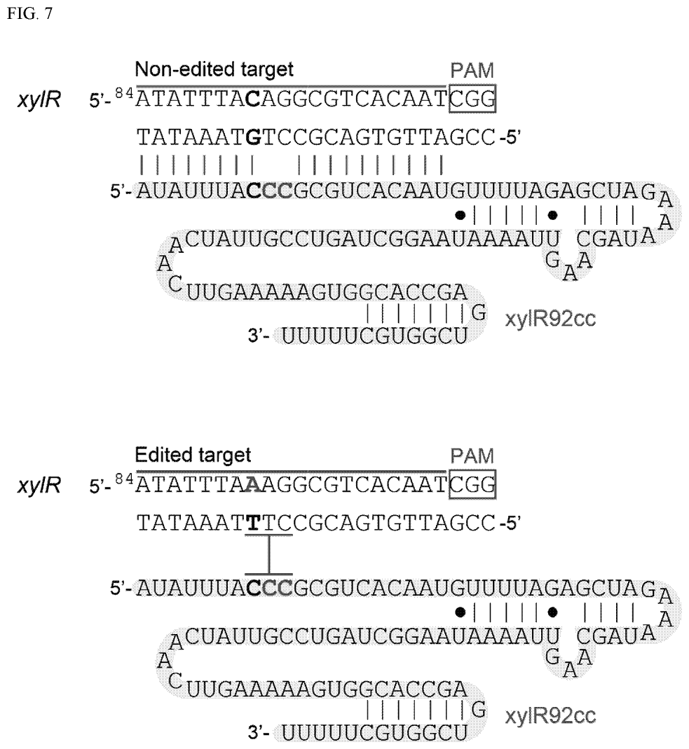

Accordingly, a xylose consumption level of a Nissle 1917 strain in which a xylR (transcription activator) single point mutation was introduced was confirmed by using the CRISPR/Cas9 including the oligonucleotide-induced mutagenesis and the mismatch guide RNA of the present invention (FIG. 7).

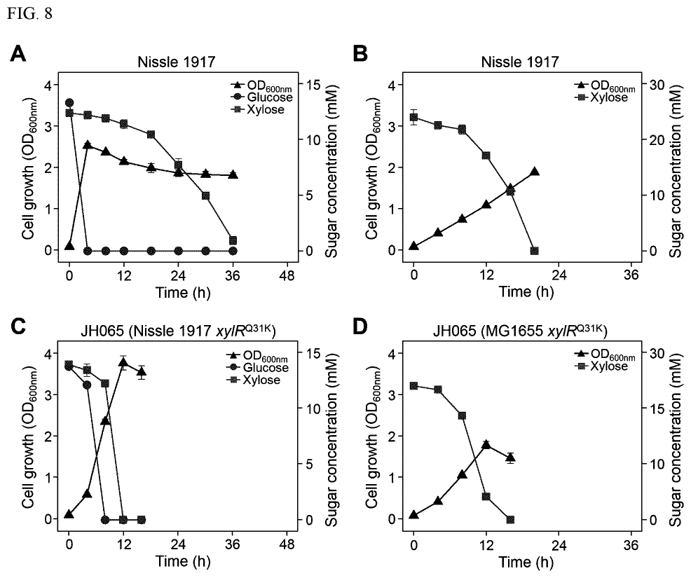

As a result, as illustrated in FIGS. 8A to 8D, the consumption rate of xylose sugar was significantly increased in the E. coli strain into which xylR single point mutation was introduced using the mismatch guide RNA/Cas9 of the present invention.

That is, under anaerobic conditions, E. coli, a genome-edited microorganism according to the present invention, more increased the consumption rate of xylose sugar at (D) than (B), and when glucose and xylose sugar were present simultaneously under anaerobic conditions, it was confirmed that the consumption rate of xylose sugar was more significantly increased at (C) than (A).

This verifies that the CRISPR/Cas9 system using the oligonucleotide-induced mutagenesis and the mismatch guide RNA of the present invention is actually operated.

Therefore, the present invention not only improves the point mutation introduction efficiency compared to a conventional CRISPR/Cas9 system, but also actually achieves a sophisticated gene editing effect of a single base unit. Accordingly, it is possible to have codons and metabolic pathways optimized for material production by correctly repairing the genomes of microbial strains in which a mutation (e.g., point mutation) has occurred in the target gene, or causing a codon change and the like. Therefore, the present invention can be variously used for the production of strains optimized for the production capacity of useful materials, and can be very useful for industrial applications related to the production of useful products.

As described above, specific parts of the present invention have been described in detail, and it will be apparent to those skilled in the art that these specific techniques are merely preferred embodiments, and the scope of the present invention is not limited thereto. Therefore, the substantial scope of the present disclosure will be defined by the appended claims and their equivalents.

1. A genome editing method based on a Clustered Regularly Interspaced Short Palindromic Repeats/CRISPR-associated protein 9 (CRISPR/Cas9) system, comprising a donor nucleic acid molecule and guide RNA that complementarily bind to a target DNA, the genome editing method comprising:
generating one or more mismatched nucleotides between the target DNA and the guide RNA sequence.

2. The method of claim 1, wherein the guide RNA is dual RNA comprising CRISPR RNA (crRNA) and transactivating crRNA (tracrRNA), or single-chain guide RNA (sgRNA) comprising a part of the crRNA and tracrRNA.

3. The method of claim 2, wherein the target DNA comprises a nucleotide of a complementary sequence to the crRNA or sgRNA and a protospacer-adjacent motif (PAM).

4. The method of claim 1, wherein the donor nucleic acid molecule has a single-stranded or double-stranded form.

5. The method of claim 1, wherein the donor nucleic acid molecule causes a genetic modification on the target DNA.

6. The method of claim 5, wherein the modification includes a substitution of one or more nucleotides, an insertion of one or more nucleotides, a deletion of one or more nucleotides, a knockout, a knockin, a replacement of an endogenous nucleic acid sequence with a homologous, orthologous, or heterologous nucleic acid sequence, or a combination thereof.

7. A genome editing composition based on a CRISPR/Cas9 system comprising a donor nucleic acid molecule and guide RNA that complementarily bind to target DNA, to generate one or more mismatched nucleotides between the target DNA and the guide RNA sequence.

8. A method for increasing genome editing efficiency based on a CRISPR/Cas9 system comprising a donor nucleic acid molecule and guide RNA that complementarily bind to target DNA, comprising:
generating one or more mismatched nucleotides between the target DNA and the guide RNA sequence.

9. A method for preparing a subject in which a target DNA is edited based on a CRISPR/Cas9 system, comprising the steps of:
(a) constructing a donor nucleic acid molecule that complementarily binds to the target DNA and causes modification on the target DNA;
(b) constructing guide RNA which complementarily binds to the target DNA and has one or more mismatched nucleotides generated to the target DNA; and
(c) contacting the donor nucleic acid molecule of step (a) and the guide RNA of step (b) into the subject to be edited, thereby two or more mismatches occur between the target DNA and the guide RNA sequence to edit the target DNA of the subject.

10. The method of claim 9, wherein the CRISPR/Cas9 system uses antibiotic-resistant selective markers.

11. A subject in which target DNA is edited, prepared by the method of claim 9.


SEQUENCE LISTING (87 sequences)

SEQ ID NO: 1 - DNA, 85 bp, Artificial Sequence
  Cas-AraC-F1
gctttttatcgcaactctctactgtttctccatacccgtttttttggatggagtgaaacgatggataagaaatactcaataggct

SEQ ID NO: 2 - DNA, 85 bp, Artificial Sequence
  KmR-AraD-R4
gtggtgccggttgctggaatcgactgacccgcctgcgcccagatggtggcgtggcgcgagtgtaggctggagctgcttcgaagtt

SEQ ID NO: 3 - DNA, 41 bp, Artificial Sequence
  Cas-KmR-OR2
gacggatccccggaattcagtcacctcctagctgactcaaa

SEQ ID NO: 4 - DNA, 41 bp, Artificial Sequence
  Cas-KmR-OF3
gctaggaggtgactgaattccggggatccgtcgacctgcag

SEQ ID NO: 5 - DNA, 30 bp, Artificial Sequence
  Ara-F1
agcagctccgaatagcgcccttccccttgc

SEQ ID NO: 6 - DNA, 28 bp, Artificial Sequence
  AraD_500dn
ccagccagaaggagacttctgtcccttg

SEQ ID NO: 7 - DNA, 41 bp, Artificial Sequence
  bet_for
aggaggttataaaaaatgagtactgcactcgcaacgctggc

SEQ ID NO: 8 - DNA, 42 bp, Artificial Sequence
  bet_rev
ttatcgtgaggatgcgtcatgctgccaccttctgctctgcgg

SEQ ID NO: 9 - DNA, 42 bp, Artificial Sequence
  rep101_for
aaggtggcagcatgacgcatcctcacgataatatccgggtag

SEQ ID NO: 10 - DNA, 44 bp, Artificial Sequence
  ara_promoter_rev
gagtgcagtactcattttttataacctccttagagctcgaattc

SEQ ID NO: 11 - DNA, 41 bp, Artificial Sequence
  galK504A
gaaaaccagtttgtaggctgaaactgcgggatcatggatca

SEQ ID NO: 12 - DNA, 41 bp, Artificial Sequence
  galK504AT
gaaaaccagtttgtaggctgatactgcgggatcatggatca

SEQ ID NO: 13 - DNA, 41 bp, Artificial Sequence
  galK503AGC
gaaaaccagtttgtaggctagcactgcgggatcatggatca

SEQ ID NO: 14 - DNA, 41 bp, Artificial Sequence
  galK504ATCA
aaaaccagtttgtaggctgatcatgcgggatcatggatcag

SEQ ID NO: 15 - DNA, 41 bp, Artificial Sequence
  galK578A
cttgctgatcgattgccgctaactggggaccaaagcagttt

SEQ ID NO: 16 - DNA, 30 bp, Artificial Sequence
  Sm_ATG_Out
gatactgggccggcaggcgctccattgccc

SEQ ID NO: 17 - DNA, 30 bp, Artificial Sequence
  Sm_TAA_Out
gcaatggagcgcctgccggcccagtatcag

SEQ ID NO: 18 - DNA, 34 bp, Artificial Sequence
  ClaI-gRNA-F
atcgatagcgagtcagtgagcgaggaagcggaag

SEQ ID NO: 19 - DNA, 35 bp, Artificial Sequence
  NcoI-gRNA-R
ccatgggaagatcctttgatcttttctacggggtc

SEQ ID NO: 20 - DNA, 43 bp, Artificial Sequence
  Cas-galK-WF
aggctgtaactgcgggatcagttttagagctagaaatagcaag

SEQ ID NO: 21 - DNA, 42 bp, Artificial Sequence
  Cas-galK-WR
tgatcccgcagttacagcctactagtattatacctaggactg

SEQ ID NO: 22 - DNA, 43 bp, Artificial Sequence
  galK505C_F
aggctgtcactgcgggatcagttttagagctagaaatagcaag

SEQ ID NO: 23 - DNA, 42 bp, Artificial Sequence
  galK505C_R
tgatcccgcagtgacagcctactagtattatacctaggactg

SEQ ID NO: 24 - DNA, 43 bp, Artificial Sequence
  galK505CC_F
aggctgtccctgcgggatcagttttagagctagaaatagcaag

SEQ ID NO: 25 - DNA, 42 bp, Artificial Sequence
  galK505CC_R
tgatcccgcagggacagcctactagtattatacctaggactg

SEQ ID NO: 26 - DNA, 43 bp, Artificial Sequence
  galK502GC_F
aggcgctaactgcgggatcagttttagagctagaaatagcaag

SEQ ID NO: 27 - DNA, 42 bp, Artificial Sequence
  galK502GC_R
tgatcccgcagttagcgcctactagtattatacctaggactg

SEQ ID NO: 28 - DNA, 43 bp, Artificial Sequence
  galK503C_F
aggctctaactgcgggatcagttttagagctagaaatagcaag

SEQ ID NO: 29 - DNA, 42 bp, Artificial Sequence
  galK503C_R
tgatcccgcagttagagcctactagtattatacctaggactg

SEQ ID NO: 30 - DNA, 43 bp, Artificial Sequence
  galK563_F
tgatcgattgccgctcactggttttagagctagaaatagcaag

SEQ ID NO: 31 - DNA, 42 bp, Artificial Sequence
  galK563_R
cagtgagcggcaatcgatcaactagtattatacctaggactg

SEQ ID NO: 32 - DNA, 43 bp, Artificial Sequence
  galK579C_F
tgatcgattgccgctccctggttttagagctagaaatagcaag

SEQ ID NO: 33 - DNA, 42 bp, Artificial Sequence
  galK579C_R
cagggagcggcaatcgatcaactagtattatacctaggactg

SEQ ID NO: 34 - DNA, 43 bp, Artificial Sequence
  galK579CA_F
tgatcgattgccgctccatggttttagagctagaaatagcaag

SEQ ID NO: 35 - DNA, 42 bp, Artificial Sequence
  galK579CA_R
catggagcggcaatcgatcaactagtattatacctaggactg

SEQ ID NO: 36 - DNA, 43 bp, Artificial Sequence
  galK577G_F
tgatcgattgccgcgcactggttttagagctagaaatagcaag

SEQ ID NO: 37 - DNA, 42 bp, Artificial Sequence
  galK577G_R
cagtgcgcggcaatcgatcaactagtattatacctaggactg

SEQ ID NO: 38 - DNA, 43 bp, Artificial Sequence
  galK576AG_F
tgatcgattgccgagcactggttttagagctagaaatagcaag

SEQ ID NO: 39 - DNA, 42 bp, Artificial Sequence
  galK576AG_R
cagtgctcggcaatcgatcaactagtattatacctaggactg

SEQ ID NO: 40 - DNA, 41 bp, Artificial Sequence
  yaaA
ttgatgaagattcctccagcgatggcgaactggtgtttaaa

SEQ ID NO: 41 - DNA, 41 bp, Artificial Sequence
  yaaA
ttgatgaagattcctccagctatggcgaactggtgtttaaa

SEQ ID NO: 42 - DNA, 41 bp, Artificial Sequence
  yaaA
ttgatgaagattcctccagccatggcgaactggtgtttaaa

SEQ ID NO: 43 - DNA, 41 bp, Artificial Sequence
  galK
tcgaaatcaccgtgccgcaagttgacactctggtagaaatc

SEQ ID NO: 44 - DNA, 41 bp, Artificial Sequence
  galK
tcgaaatcaccgtgccgcaatttgacactctggtagaaatc

SEQ ID NO: 45 - DNA, 41 bp, Artificial Sequence
  galK
tcgaaatcaccgtgccgcaacttgacactctggtagaaatc

SEQ ID NO: 46 - DNA, 41 bp, Artificial Sequence
  ybdG
attgggttaaccaccgtcaaggtgcgtaactgggacaatac

SEQ ID NO: 47 - DNA, 41 bp, Artificial Sequence
  ybdG
attgggttaaccaccgtcaatgtgcgtaactgggacaatac

SEQ ID NO: 48 - DNA, 41 bp, Artificial Sequence
  ybdG
attgggttaaccaccgtcaacgtgcgtaactgggacaatac

SEQ ID NO: 49 - DNA, 41 bp, Artificial Sequence
  moeA
cggtggtgatgcaggagcaggctgaacaaatggacaatggc

SEQ ID NO: 50 - DNA, 41 bp, Artificial Sequence
  moeA
cggtggtgatgcaggagcagtctgaacaaatggacaatggc

SEQ ID NO: 51 - DNA, 41 bp, Artificial Sequence
  moeA
cggtggtgatgcaggagcagcctgaacaaatggacaatggc

SEQ ID NO: 52 - DNA, 41 bp, Artificial Sequence
  galK
gaaaaccagtttgtaggctggaactgcgggatcatggatca

SEQ ID NO: 53 - DNA, 41 bp, Artificial Sequence; identical to SEQ ID NO: 11
  galK

SEQ ID NO: 54 - DNA, 41 bp, Artificial Sequence
  galK
gaaaaccagtttgtaggctgcaactgcgggatcatggatca

SEQ ID NO: 55 - DNA, 41 bp, Artificial Sequence
  ydcO
cattaattgtatttactggagtgctggcactggttttttcc

SEQ ID NO: 56 - DNA, 41 bp, Artificial Sequence
  ydcO
cattaattgtatttactggaatgctggcactggttttttcc

SEQ ID NO: 57 - DNA, 41 bp, Artificial Sequence
  ydcO
cattaattgtatttactggactgctggcactggttttttcc

SEQ ID NO: 58 - DNA, 41 bp, Artificial Sequence
  ydiU
ttgtagtgccatcagcaagcggttgttcgccgagtaaaatg

SEQ ID NO: 59 - DNA, 41 bp, Artificial Sequence
  ydiU
ttgtagtgccatcagcaagctgttgttcgccgagtaaaatg

SEQ ID NO: 60 - DNA, 41 bp, Artificial Sequence
  ydiU
ttgtagtgccatcagcaagccgttgttcgccgagtaaaatg

SEQ ID NO: 61 - DNA, 41 bp, Artificial Sequence
  preT
ccagacccgcgacccgcaaggctttgctgctggcgatattg

SEQ ID NO: 62 - DNA, 41 bp, Artificial Sequence
  preT
ccagacccgcgacccgcaagactttgctgctggcgatattg

SEQ ID NO: 63 - DNA, 41 bp, Artificial Sequence
  preT
ccagacccgcgacccgcaagcctttgctgctggcgatattg

SEQ ID NO: 64 - DNA, 41 bp, Artificial Sequence
  ypdA
cggtggcgattactaataccaatcatgtgctggcctatgtt

SEQ ID NO: 65 - DNA, 41 bp, Artificial Sequence
  ypdA
cggtggcgattactaatacctatcatgtgctggcctatgtt

SEQ ID NO: 66 - DNA, 41 bp, Artificial Sequence
  ypdA
cggtggcgattactaatacccatcatgtgctggcctatgtt

SEQ ID NO: 67 - DNA, 41 bp, Artificial Sequence
  proX
cgataccaaactgccgaatgatgcgaattatggcttcccgg

SEQ ID NO: 68 - DNA, 41 bp, Artificial Sequence
  proX
cgataccaaactgccgaatgttgcgaattatggcttcccgg

SEQ ID NO: 69 - DNA, 41 bp, Artificial Sequence
  proX
cgataccaaactgccgaatgctgcgaattatggcttcccgg

SEQ ID NO: 70 - DNA, 41 bp, Artificial Sequence
  fau
tggtcgcctttgatgagtacagtcagcgcctgggaatgggc

SEQ ID NO: 71 - DNA, 41 bp, Artificial Sequence
  fau
tggtcgcctttgatgagtactgtcagcgcctgggaatgggc

SEQ ID NO: 72 - DNA, 41 bp, Artificial Sequence
  fau
tggtcgcctttgatgagtaccgtcagcgcctgggaatgggc

SEQ ID NO: 73 - DNA, 41 bp, Artificial Sequence
  yhbU
gttacgcccgttggcagcgcaccgtggatatggcggcgcag

SEQ ID NO: 74 - DNA, 41 bp, Artificial Sequence
  yhbU
gttacgcccgttggcagcgctccgtggatatggcggcgcag

SEQ ID NO: 75 - DNA, 41 bp, Artificial Sequence
  yhbU
gttacgcccgttggcagcgccccgtggatatggcggcgcag

SEQ ID NO: 76 - DNA, 41 bp, Artificial Sequence
  galK
cttgctgatcgattgccgctgactggggaccaaagcagttt

SEQ ID NO: 77 - DNA, 41 bp, Artificial Sequence; identical to SEQ ID NO: 15
  galK

SEQ ID NO: 78 - DNA, 41 bp, Artificial Sequence
  galK
cttgctgatcgattgccgcttactggggaccaaagcagttt

SEQ ID NO: 79 - DNA, 41 bp, Artificial Sequence
  mnmE
tatcgtagcccaggccacgcgtccgggacgtggcggcgttg

SEQ ID NO: 80 - DNA, 41 bp, Artificial Sequence
  mnmE
tatcgtagcccaggccacgcatccgggacgtggcggcgttg

SEQ ID NO: 81 - DNA, 41 bp, Artificial Sequence
  mnmE
tatcgtagcccaggccacgcttccgggacgtggcggcgttg

SEQ ID NO: 82 - DNA, 41 bp, Artificial Sequence
  thiH
ccatgagtaatcgcatcaagggcaaaacgctggatgaagcg

SEQ ID NO: 83 - DNA, 41 bp, Artificial Sequence
  thiH
ccatgagtaatcgcatcaagagcaaaacgctggatgaagcg

SEQ ID NO: 84 - DNA, 41 bp, Artificial Sequence
  thiH
ccatgagtaatcgcatcaagtgcaaaacgctggatgaagcg

SEQ ID NO: 85 - DNA, 41 bp, Artificial Sequence
  yjhF
ccccggcccgactgttatcggcagggaatatggcgctgatg

SEQ ID NO: 86 - DNA, 41 bp, Artificial Sequence
  yjhF
ccccggcccgactgttatcgacagggaatatggcgctgatg

SEQ ID NO: 87 - DNA, 41 bp, Artificial Sequence
  yjhF
ccccggcccgactgttatcgtcagggaatatggcgctgatg